(19) United States
(12) Patent Application Publication
(10) Pub. No.: US 2024/0360520 A1
(43) Pub. Date: Oct. 31, 2024

(54) COMPOSITE PRIMER SET AND KIT FOR IDENTIFYING POLYMORPHIC MARKERS OF CATTLE AND APPLICATIONS THEREOF

(71) Applicant: Fudan University, Shanghai (CN)

(72) Inventors: SUHUA ZHANG, Shanghai (CN); CHENGTAO LI, Shanghai (CN)

(21) Appl. No.: 18/354,652

(22) Filed: Jul. 19, 2023

(30) Foreign Application Priority Data
Apr. 25, 2023 (CN) 2023104557225

(51) Int. Cl. C12Q 1/6888

(52) U.S. Cl. CPC C12Q 1/6888; C12Q 2600/156; C12Q 2600/16

(57) ABSTRACT

A primer composition, a kit and a method for detecting polymorphic STR markers based on capillary electrophoresis and applications thereof are provided, which are used to simultaneously amplify 13 STR loci on cattle genome. The primer composition includes one or more pairs of primers with sequences as shown in SEQ ID NO: 1˜26. Developmental validation indicated that the kit is of high sensitivity, fine specificity, strong stability and high accuracy. This provides an effective tool for species identification, individual identification and parental analysis of cattle samples in civil disputes and illegal crimes.

### TECHNICAL FIELD

The disclosure is related to the field of molecular identification, particularly to a composite primer set and a kit for identifying polymorphic markers of cattle and applications thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 23031THXT-USP1-SL.xml. The XML file is 23,982 bytes; is created on Jul. 17, 2023; and is being submitted electronically via EFS-Web.

### BACKGROUND

In recent years, with a significant increase in civil and criminal cases related to animals, there is an increasing need to use the DNA technology for species identification, intra-species genealogical relationship analysis and individual identification. Cattle, as one of the most common species in animal husbandry, are also the main object of many civil disputes and crimes.

In modern pasture breeding, artificial insemination and embryo transfer have been widely used in the breeding of dairy and beef cattle. In this situation, it is of great significance for genetic breeding to establish and improve an animal pedigree map in order to clarify the genetic relationship between animals and conduct effective breeding retention. Previously, Visscher et al. have studied 568 British dairy cattle and found a genealogical error rate of 8.8% among the cattle hair samples, a genealogical error rate of 13.1% among the cattle milk samples, and a total genealogical error rate of 10% during the overall pedigree recording. Geldrmann et al. have found a genealogical error rate of 13% among German dairy cattle, with it resulting in a decrease of 8% to 16.9% in yields of milk and milk fat. The results of the above studies illustrate that in some cases, the blood relations among cattle individuals cannot be confirmed, which obstructs the development of breeding. In addition, during the production and quality testing of frozen semen from herd bulls, some vendors intentionally engage in shoddy illegal or even criminal activities. With the development of contemporary society, growth of population size and general increase of social wealth, meat consumption of different animals increases year by year. Meat fraud and adulteration occur around the world from time to time, which not only seriously undermine market order, but also increase risks of religious and ethnic conflicts. Therefore, taking effective measures (such as developing accurate meat species identification technology) to ensure the authenticity of meat products is of great and practical significance.

Short tandem repeat (STR) is regarded as an oligonucleotide sequence with core sequence of two to six bases in tandem. Because of its high sensitivity, strong identification ability, fine species specificity, high accuracy and easy standardization, STR is widely used in the field of forensic science on individual identification, kinship identification and investigation of population, while it has also become the most widely used genetic marker in forensic science. Accordingly, researches on animal STR loci have been gradually developed. In 2012, Ogden et al. constructed a detection system of 15 STR loci for individual identification and kinship identification of dogs. In 2013, Rosa et al. conducted the kinship identification of Valley Belle sheep using 24 STR loci. In 2014, Thermo Fisher Scientific Inc of the United States launched a commercialized kit for cattle (referred to as Stock Marks® Kits for Cattle) containing 11 STR loci. However, its genotyping effect of each STR locus was poor, and the kit was quite expensive.

In addition, most of the core sequences of cattle STR loci involved in the related studies are dinucleotide repeats which will generate high stutter peaks during amplification. In this case, the above-mentioned core sequences are not conducive to the application of forensic identification. And there are fewer STR loci with tetranucleotide repeats, resulting in a lower cumulative non-paternity exclusion rate for kinship identification. Therefore, the development of a multiple kit containing effective cattle STR loci plays an important role in species identification, individual identification and parental identification of cattle samples in civil disputes and illegal crimes involving cattle.

### SUMMARY

An objective of the disclosure is to provide a composite primer set and an accordingly kit for species identification, individual identification and kinship analysis of cattle samples.

To achieve the above objective, the disclosure provides technical solutions as follows.

In a first aspect of the disclosure, a composite primer set for identifying polymorphic markers of cattle is provided, including:
a nucleotide sequence of an upstream primer for amplifying TGLA126 gene shown in SEQ ID NO: 1, and a nucleotide sequence of a downstream primer for amplifying the TGLA126 gene shown in SEQ ID NO: 2;
a nucleotide sequence of an upstream primer for amplifying BT66 gene shown in SEQ ID NO: 3, and a nucleotide sequence of a downstream primer for amplifying the BT66 gene shown in SEQ ID NO: 4;
a nucleotide sequence of an upstream primer for amplifying BT165 gene shown in SEQ ID NO: 5, and a nucleotide sequence of a downstream primer for amplifying the BT165 gene shown in SEQ ID NO: 6;
a nucleotide sequence of an upstream primer for amplifying ETH10 gene shown in SEQ ID NO: 7, and a nucleotide sequence of a downstream primer for amplifying the ETH10 gene shown in SEQ ID NO: 8;
a nucleotide sequence of an upstream primer for amplifying CSM0113 gene shown in SEQ ID NO: 9, and a nucleotide sequence of a downstream primer for amplifying the CSM0113 gene shown in SEQ ID NO: 10;
a nucleotide sequence of an upstream primer for amplifying INRA005 gene shown in SEQ ID NO: 11, and a nucleotide sequence of a downstream primer for amplifying the INRA005 gene shown in SEQ ID NO: 12;
a nucleotide sequence of an upstream primer for amplifying BT54 gene shown in SEQ ID NO: 13, and a nucleotide sequence of a downstream primer for amplifying the BT54 gene shown in SEQ ID NO: 14;
a nucleotide sequence of an upstream primer for amplifying BT61 gene shown in SEQ ID NO: 15, and a nucleotide sequence of a downstream primer for amplifying the BT54 gene shown in SEQ ID NO: 16;
a nucleotide sequence of an upstream primer for amplifying INRA023 gene shown in SEQ ID NO: 17, and a nucleotide sequence of a downstream primer for amplifying the INRA023 gene shown in SEQ ID NO: 18;
a nucleotide sequence of an upstream primer for amplifying BM2113 gene shown in SEQ ID NO: 19, and a nucleotide sequence of a downstream primer for amplifying the BM2113 gene shown in SEQ ID NO: 20;
a nucleotide sequence of an upstream primer for amplifying G18833 gene shown in SEQ ID NO: 21, and a nucleotide sequence of a downstream primer for amplifying the G18833 gene shown in SEQ ID NO: 22;
a nucleotide sequence of an upstream primer for amplifying UMN0929 gene shown in SEQ ID NO: 23, and a nucleotide sequence of a downstream primer for amplifying the UMN0929 gene shown in SEQ ID NO: 24; and a nucleotide sequence of an upstream primer for amplifying INRA063 gene shown in SEQ ID NO: 25, and a nucleotide sequence of a downstream primer for amplifying the INRA063 gene shown in SEQ ID NO: 26.

In a second aspect of the disclosure, a kit for identifying polymorphic markers of cattle is provided. In an embodiment, the kit includes the composite primer set for identifying polymorphic markers of cattle described above.

In a third aspect of the disclosure, a method for detecting 13 STR loci of cattle using the primer set or the kit is provided. The method includes an adoption of the primer set or the kit in a polymerase chain reaction (PCR) system to perform multiplex PCR amplification on cattle samples to obtain PCR amplification products. Genotyping detection is then performed on the above PCR amplification products.

In an embodiment, final reaction concentrations of each pair of the primers in the primer set are as follows:
the upstream primer and the downstream primer of the TGLA126 gene each is 1.5 micromoles per liter (μM);
the upstream primer and the downstream primer of the BT66 gene each is 0.6 μM;
the upstream primer and the downstream primer of the BT165 gene each is 0.6 μM;
the upstream primer and the downstream primer of the ETH10 gene each is 1 μM;
the upstream primer and the downstream primer of the CSM0113 gene each is 2.5 μM;
the upstream primer and the downstream primer of the INRA005 gene each is 2.5 UM;
the upstream primer and the downstream primer of the BT54 gene each is 1.5 μM;
the upstream primer and the downstream primer of the BT61 gene each is 2.25 μM;
the upstream primer and the downstream primer of the INRA023 gene each is 2.5 μM;
the upstream primer and the downstream primer of the BM2113 gene each is 3 μM;
the upstream primer and the downstream primer of the G18833 gene each is 2.5 μM;
the upstream primer and the downstream primer of the UMN0929 gene each is 4 μM; and
the upstream primer and the downstream primer of the INRA063 gene each is 5 μM.

In an embodiment, the PCR system further includes: 2×Multiplex PCR Master Mix, 5×Q-Solution, 10×Primer mixture, deionized water, and genomic deoxyribonucleic acid (DNA).

In an embodiment, reaction conditions of the multiplex PCR amplification include: pre-denaturation at 95 degrees Celsius (C) for 15 minutes; denaturation at 94° C. for 30 seconds, annealing at 57° C. for 90 seconds, extension at 72° C. for 90 seconds, repeat for 30 cycles; then final extension at 60° C. for 60 minutes and heat preservation at 4° C.

In an embodiment, at least one primer in each pair of the primers in the primer set is labeled with a fluorescent dye and the fluorescent dye is selected among 6-carboxyfluorescein (6FAM), hexachlorofluorescein (HEX), carboxytetramethylrhodamine (TAMRA), and carboxy-X-rhodamine (ROX).

In a fourth aspect of the disclosure, the method in which the primer set or the kit is adopted is applied for species identification, individual identification, and kinship analysis on cattle.

The disclosure has the advantages as follows.

1. The disclosure simultaneously amplifies 13 cattle STR loci, and provides the kit containing the above fluorescent modified polymorphic genetic marker with the multiplex amplification system.

2. The disclosure provides effective methods for further research on cattle STR loci, establishment of the cattle STR database, as well as detection and identification of the cattle samples in criminal cases.

3. The disclosure establishes a forensic detection kit with high sensitivity, fine specificity and strong stability. It also provides an effective detection tool for species identification, individual identification, and kinship analysis on cattle, which is of great significance for meeting the needs of judicial expertise for cattle.

### BRIEF DESCRIPTION OF DRAWINGS

In order to illustrate embodiments of disclosure and technical solutions in related art more clearly, brief introductions will be made to attached drawings needed in the embodiments below. Apparently, the attached drawings provided below are merely some embodiments of the disclosure. For those skilled in the art, other drawings can be obtained from these attached drawings without any creative effort.

FIG. 1 illustrates a genotyping profile of cattle DNA samples according to embodiment 1 of the disclosure.

FIG. 2 illustrates sensitivity results of an amplification method for the cattle DNA samples according to the embodiment 1 of the disclosure.

FIGS. 3A-3D illustrate stability results of the amplification method for cattle DNA samples according to the embodiment 1 of the disclosure. Specifically, FIG. 3A illustrates average allele detection rates of 1 nanogram (abbreviated as 1 ng) of cattle DNA samples treated with different concentrations of heme. FIG. 3B illustrates average allele detection rates of 1 ng cattle DNA samples treated with different concentrations of humic acid. FIG. 3C illustrates average allele detection rates of 1 ng cattle DNA samples treated with different concentrations of urea. FIG. 3D illustrates average allele detection rates of 1 ng cattle DNA samples treated with different concentrations of indigo.

### DETAILED DESCRIPTION OF EMBODIMENTS

Various illustrated embodiments of the disclosure are now described in detail. However, the detailed description should not be considered a limitation of the disclosure, but instead comprehended as a more detailed description of certain aspects, features and embodiments of the disclosure.

It is to be understood that the terms described in the disclosure are only intended to describe the illustrated embodiments but not to limit the disclosure. Further, with respect to the value ranges in the disclosure, it is to be understood that intermediate values between upper and lower limits of the value ranges are also specifically disclosed. Sub-ranges between stated values or intermediate values within stated ranges, and other stated values or intermediate values within the ranges are also included in the disclosure. The upper and lower limits of these sub-ranges may be independently included or excluded from the scope of the disclosure.

Unless otherwise indicated, all technical and scientific terms used herein are of the same meanings as commonly comprehended by those skilled in the related art described herein. Although the disclosure is only to describe the illustrated methods and materials, any methods and materials similar or equivalent to those described herein may also be used in the implementation or testing of the disclosure. All references in the summary are incorporated for the purpose of disclosing and describing the methods and/or materials associated. In the case of conflict, the contents of the disclosure shall prevail.

The terms “comprises”, “includes”, “has”, “contains”, etc., as used herein, are open-ended terms. In other words, these terms are meant to include but not limit to.

The disclosure involves a multiplex amplification system with fluorescence-modified polymorphic genetic markers and its application. The multiplex amplification system includes amplification primers for 13 STR loci of cattle. Specifically, the 13 STR loci are TGLA126, BT66, BT165, ETH10, CSSM0113, INRA005, BT54, BT61, INRA023, BM2113, G18833, UMN0929, and INRA063. Corresponding information-sequences of amplification primers, final reaction concentrations of primers and fluorescence markers applied are illustrated in Table 1.

Table 1 illustrates sequences of amplification primers, final reaction
concentrations of primers and fluorescence markers applied.

| | | | Final | |
|---|---|---|---|---|
| Name of | | | concentration | Fluorescence |
| genetic | Serial | | of primers | modifi- |
| locus | number | Primer sequence (5′-3′) | (μM) | cation |
| TGLA126 | SEQ | TTGGTCCTCTATTCTCTGAATATTCC | 1.50 | 6FAM |
| | ID | | | |
| | NO: 1 | | | |
| | SEQ | CTAATTTAGAATGAGAGAGGCTTCT | 1.50 | |
| | ID | | | |
| | NO: 2 | | | |
| BT66 | SEQ | GCAGCATTTCTTTGGCTGTAAA | 0.60 | 6FAM |
| | ID | | | |
| | NO: 3 | | | |
| | SEQ | GTGGTTGCCCGTTCATTA | 0.60 | |
| | ID | | | |
| | NO: 4 | | | |
| BT165 | SEQ | GGATCTTCCCGATAACCC | 0.60 | 6FAM |
| | ID | | | |
| | NO: 5 | | | |
| | SEQ | GTAGCAGCAGTAGCAGTTC | 0.60 | |
| | ID | | | |
| | NO: 6 | | | |
| ETH10 | SEQ | ACCTGGGTAGTCGGAG | 1.00 | 6FAM |
| | ID | | | |
| | NO: 7 | | | |
| | SEQ | GGGACCATCCATTCTGC | 1.00 | |
| | ID | | | |
| | NO: 8 | | | |
| CSM0113 | SEQ | AGCAAGACAGGTGTTTCAA | 2.50 | HEX |
| | ID | | | |
| | NO: 9 | | | |
| | SEQ | CGACTCTGGGGATGATGTA | 2.50 | |
| | ID | | | |
| | NO: | | | |
| | 10 | | | |
| INRA005 | SEQ | GGGAATCTGTGGAGGAG | 2.50 | HEX |
| | ID | | | |
| | NO: | | | |
| | 11 | | | |
| | SEQ | CAGGCATACCCTACACC | 2.50 | |
| | ID | | | |
| | NO: | | | |
| | 12 | | | |
| BT54 | SEQ | GTAAGTGACTGCCAGAAACC | 1.50 | HEX |
| | ID | | | |
| | NO: | | | |
| | 13 | | | |
| | SEQ | AACAGGAACCAGCATGAAC | 1.50 | |
| | ID | | | |
| | NO: | | | |
| | 14 | | | |
| BT61 | SEQ | TAATGAGCGAGTGAAACAACG | 2.25 | HEX |
| | ID | | | |
| | NO: | | | |
| | 15 | | | |
| | SEQ | CAGTGTAAACTTGCCTGTCTT | 2.25 | |
| | ID | | | |
| | NO: | | | |
| | 16 | | | |
| INRA023 | SEQ | AACTACAGGGTGTTAGATGAAC | 2.50 | TAMRA |
| | ID | | | |
| | NO: | | | |
| | 17 | | | |
| | SEQ | CAATGGGTCAGAGTAGAGC | 2.50 | |
| | ID | | | |
| | NO: | | | |
| | 18 | | | |
| BM2113 | SEQ | TGTTTATCATCCAAGGTGCCA | 3.00 | TAMRA |
| | ID | | | |
| | NO: | | | |
| | 19 | | | |
| | SEQ | CTCCCAGATCAATCCAAGA | 3.00 | |
| | ID | | | |
| | NO: | | | |
| | 20 | | | |
| G18833 | SEQ | GATCACTTGAGCCAATTCCAATA | 2.50 | TAMRA |
| | ID | | | |
| | NO: | | | |
| | 21 | | | |
| | SEQ | GCAACACCTACCGTCTTACT | 2.50 | |
| | ID | | | |
| | NO: | | | |
| | 22 | | | |
| UMN0929 | SEQ | ACCAGCTGATACACAAGTGC | 4.00 | ROX |
| | ID | | | |
| | NO: | | | |
| | 23 | | | |
| | SEQ | GGTCAGAGAATGAAACAGAG | 4.00 | |
| | ID | | | |
| | NO.24 | | | |
| INRA063 | SEQ | TGTAGCTGGCTTACTTACGA | 5.00 | ROX |
| | ID | | | |
| | NO: | | | |
| | 25 | | | |
| | SEQ | AAGGTGACCACAAAGGGATT | 5.00 | |
| | ID | | | |
| | NO: | | | |
| | 26 | | | |

In an illustrated embodiment of the disclosure, the primer set includes primers with nucleotide sequences SEQ ID NO: 1 to SEQ ID NO: 26.

In addition, the kit includes the above-described composite primer set for identifying polymorphic markers of cattle, 2×Multiplex PCR Master Mix (providing a final concentration of 3 millimoles per liter abbreviated as mM MgCl₂), 5×Q-Solution, and deionized water. In an embodiment, the Multiplex PCR Master Mix and the 5×Q-Solution are available from QIAGEN Multiplex PCR Kit, Qiagen, Germany.

The disclosure is described below in detail, specifically by means of the illustrated embodiments and attached figures for a better understanding of the disclosure. Nevertheless, the following embodiments should not limit the scope of the disclosure.

The methods in the embodiments are conventional if no other specific indication is given. The reagents used are commercially available ones or ones prepared by conventional methods if no other specific indication is given.

### Embodiment 1

The method used for identifying polymorphic markers of cattle includes the following steps.

### 1. Screening of STR Loci of Cattle for Application in Forensic Medicine

The disclosure refers to the STR combinations recommended jointly by Food and Agriculture Organization of the United Nations (FAO) and International Society for Animal Genetics (ISAG) for research on animal genetic diversity and reports, which is adopted here for screening of cattle STR loci. The screening should abide by the following principles: large number of alleles, high polymorphism, no linkage among genetic markers, and distribution of genetic markers on different chromosomes.

Finally, 13 optimal cattle STR loci are selected for kit development, including 8 commonly used cattle STR loci and 5 STR loci with tetranucleotide repeats and fine polymorphism (also referred to as 5 four-base repeat STR loci with fine polymorphism). The loci mentioned here are TGLA126, BT66, BT165, ETH10, CSM011, INRA005, BT54, BT61, INRA023, BM2113, G18833, UMN0929, and INRA063.

### 2. Design of the Amplification Primers

A composite amplification detection kit containing five fluorescent markers (FAM, HEX, TAMRA, ROX and ORG) is established. The internal standards of molecular weights are labeled with the ORG fluorescent marker (ORG-500).

The genetic markers described in the embodiment, their corresponding amplification primer sequences, the fluorescence markers applied and the final reaction concentrations are illustrated in Table 1.

### 3. Construction and Optimization of a Multiplex Polymerase Chain Reaction (PCR) Amplification System

PCR reaction conditions of the constructed multiplex amplification typing system are adjusted and optimized, including primer concentration, DNA template quantity, annealing temperature, and number of PCR cycles, to obtain balanced and stable PCR product typing results and achieve multiplex amplification of the 13 STR loci.

The described multiplex PCR amplification system containing 13 cattle STR loci is illustrated in Table 2, in which DNA needs to be extracted from venous blood, buccal swabs (also referred to as oral swabs), tissues, hair, etc. of cattle.

Table 2 illustrates the multipleX PCR amplification system for 13 cattle STR loci.

| | Volume (microliter |
|---|---|
| Ingredients | abbreviated as μL) |
| 2 × Multiplex PCR Master Mix | 7.5 |
| 5 × Q-Solution | 1.5 |
| Deionized water | 4.0 |
| Primer mixture (10 micromoles | 1.0 |
| per liter, abbreviated as μM) | |
| DNA (1 nanogram per microliter, | 1.0 |
| abbreviated as ng/μL) | |
| Total volume | 15 |

The reaction system can receive fine results on various reaction thermal cyclers (such as ABI 9700, ABI 9600, ABI 2720 of Applied Biosystems and Bio-Rad iCycler) with the following procedures: pre-denaturation at 95 degrees Celsius (C) for 15 minutes; denaturation at 94° C. for 30 seconds, annealing at 57° C. for 70 seconds, and extension at 72° C. for 70 seconds, repeat for 30 cycles; then final extension at 60° C. for 60 minutes; and heat preservation at 4° C. The volume of 1.0 of the primer mixture (10 μM) illustrated in Table 2 means that 26 primers are formulated into the primer mixture according to the final concentration of primers illustrated in Table 1, and 1.0 μL of it is taken.

### 4. Establishment of an Analytical Method and Detection for the Multiplex Amplification Primers

A spectral correction file is established (with system such as Matrix) for 3100 series, 3130 series, or 3500 series genetic analyzers. When adding samples into capillary electrophoresis, 1 μL of the PCR amplification primers is mixed with 8.5 L of formamide and 0.5 μL of the molecular weight internal standard (ORG-500 Size Standard); the mixture is then denatured at 95° C. for 3 minutes, cooled on ice for 3 minutes; later, genotyping detection is performed on the 13 STR loci by the above types of genetic analyzer. Electrophoretic migration parameters of different alleles of respective loci are obtained by capillary electrophoresis. On this basis, corresponding Bin files and Panel files are written according to format requirements of GeneMapperID v3.2.1 software. Thereby the electrophoretic analysis method is established.

### Embodiment 2

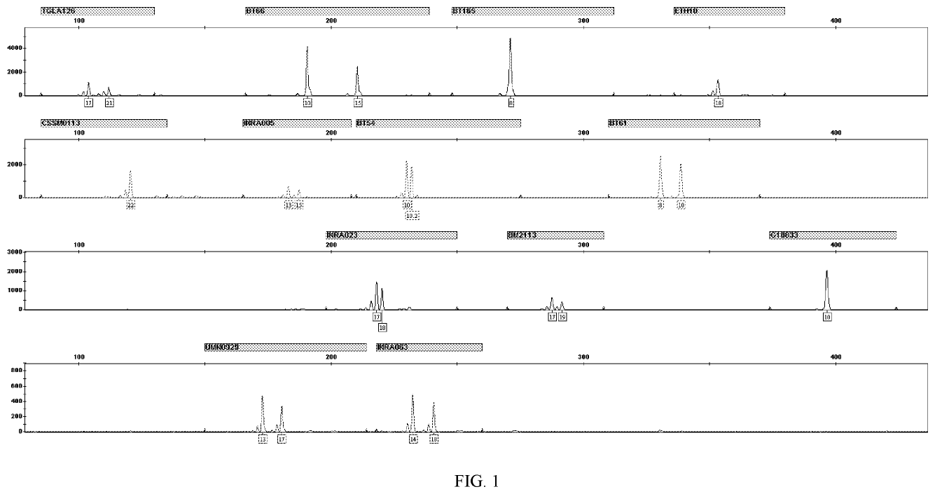

In embodiment 2, the sample DNA of one head of cattle is detected with the above method to identify polymorphic markers of the cattle, including the following steps:
(1) collecting venous blood, oral swabs and hair samples of the cattle;
(2) extracting and quantifying DNA with DNeasy Blood&Tissue Kit (available from German Qiagen Biological Company) and Qubit dsDNA HS Assay Kit (available from Thermo Fisher of the United States);
(3) using the kit constructed in embodiment 1 to perform multiplex amplification of the 13 STR loci on the obtained DNA. The genotyping map of the cattle sample DNA is shown in FIG. 1. According to the genotyping map in FIG. 1, the genotyping results of the cattle sample at 13 STR loci can be obtained.

### Embodiment 3

In embodiment 3, the DNA sample in embodiment 2 is replaced, and the total number of replacements is 95. The remaining steps are the same as embodiment 2. The results show that all samples have obtained effective amplification primers on the loci provided by the disclosure.

### Embodiment 4

Embodiment 4 is to verify the forensic work results of the method provided in embodiment 1. Specific experiments and results are as follows.

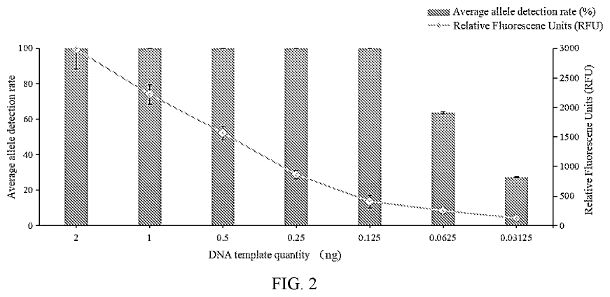

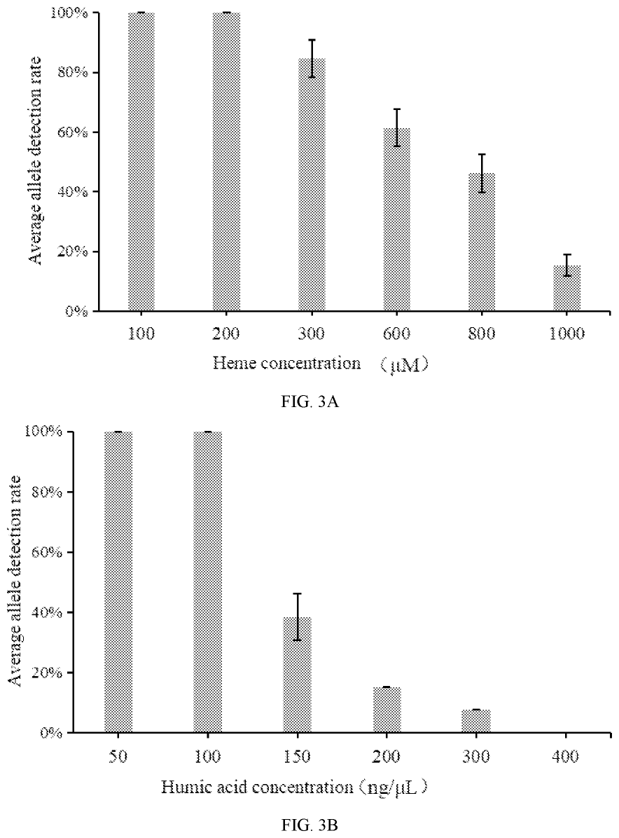

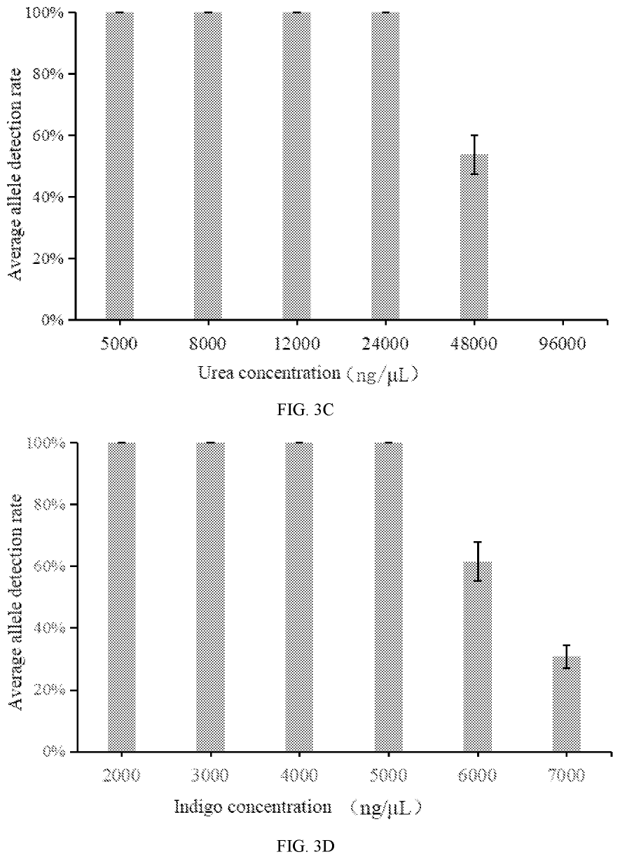

According to the requirements of Scientific Working Group for DNA Analysis Methods (SWGDAM), the multiplex amplification system and its kit constructed in embodiment 1 are tested for their identity, sensitivity, species specificity, stability, and forensic parameters calculation. The results of sensitivity study of the amplification method for sample in embodiment 2 are shown in FIG. 2. In addition, FIGS. 3A to 3D illustrate the results of stability study of the amplification method.

The same genotyping results have been obtained from oral swabs, muscles, hair, and blood samples of the same individual cattle. The results demonstrate that the multiplex amplification system and its kit constructed in embodiment 1 are of fine tissue identity. Furthermore, the multiplex amplification system and its kit are of high sensitivity and can still obtain complete genotyping map of STR loci when the DNA template amount is as low as 125 picograms (pg). Moreover, their species specificity allows them to amplify only cattle DNA effectively; they are of fine stability and tolerance to various PCR inhibitors; and they are suitable for DNA genotyping of various types of cattle samples.

The genotyping results of 96 cattle samples in embodiment 2 and embodiment 3 illustrate that 109 alleles are observed at the 13 STR loci; the genotype frequency distribution is consistent with Hardy-Weinberg equilibrium (HWE); the allele frequency of each locus is in the range of 0.0052 and 0.7604; and the forensic parameters are shown in Table 3. The 13 loci are of high discrimination power (also referred to as individual identification probability) and non-paternity exclusion probability. The cumulative discrimination power obtained here is 0.99999999997 and the cumulative non-paternity exclusion probability here is 0.99992666846 (calculation formulas refer to “Specification of Individual Identification” SF/ZJD0105012-2018 and “Specification of Parentage Testing” GB/T 37223-2018).

Table 3 illustrates the forensic parameters of 13 STR loci of cattle (n=96).

| | Number | | | Polymorphism | Individual | Non-paternity |
|---|---|---|---|---|---|---|
| Genetic | of | Heterozygosity | Heterozygosity | information | identification | exclusion |
| locus | alleles | observation | expectation | content | probability | probability |
| TGLA126 | 6 | 0.729 | 0.651 | 0.599 | 0.819 | 0.475 |
| BT66 | 10 | 0.823 | 0.811 | 0.778 | 0.927 | 0.642 |
| BT165 | 4 | 0.406 | 0.376 | 0.318 | 0.549 | 0.118 |
| ETH10 | 12 | 0.708 | 0.713 | 0.670 | 0.878 | 0.441 |
| CSM0113 | 11 | 0.969 | 0.777 | 0.742 | 0.858 | 0.937 |
| INRA005 | 6 | 0.604 | 0.672 | 0.602 | 0.817 | 0.296 |
| BT54 | 8 | 0.656 | 0.661 | 0.600 | 0.808 | 0.364 |
| BT61 | 6 | 0.594 | 0.566 | 0.488 | 0.723 | 0.283 |
| INRA023 | 12 | 0.875 | 0.816 | 0.745 | 0.941 | 0.934 |
| BM2113 | 9 | 0.823 | 0.838 | 0.814 | 0.947 | 0.242 |
| G18833 | 4 | 0.490 | 0.517 | 0.462 | 0.730 | 0.179 |
| UMN0929 | 14 | 0.781 | 0.759 | 0.732 | 0.914 | 0.565 |
| INRA063 | 7 | 0 531 | 0 535 | 0 479 | 0 722 | 0 216 |

From the above embodiments, it can be seen that the primer set, the kit, and the method of disclosure, which contains fluorescent markers and can simultaneously detect 13 STR loci of cattle samples, can provide a new type of detection method for species identification, individual identification, kinship identification, and STR database establishment in forensic research involving cattle.

Without deviating from the scope or spirit of the disclosure, it is evident to those skilled in the related art that various improvements and alternations can be made to the illustrated embodiments described in the disclosure. The other embodiments that can be obtained from the description of the disclosure are apparent to those skilled. The description and the embodiments of the disclosure are only exemplary.

What is claimed is:

1. A composite primer set for identifying polymorphic markers of cattle, comprising:
a nucleotide sequence of an upstream primer for amplifying TGLA126 gene shown in SEQ ID NO: 1, and a nucleotide sequence of a downstream primer for amplifying the TGLA126 gene shown in SEQ ID NO: 2;
a nucleotide sequence of an upstream primer for amplifying BT66 gene shown in SEQ ID NO: 3, and a nucleotide sequence of a downstream primer for amplifying the BT66 gene shown in SEQ ID NO: 4;
a nucleotide sequence of an upstream primer for amplifying BT165 gene shown in SEQ ID NO: 5, and a nucleotide sequence of a downstream primer for amplifying the BT165 gene shown in SEQ ID NO: 6;
a nucleotide sequence of an upstream primer for amplifying ETH10 gene shown in SEQ ID NO: 7, and a nucleotide sequence of a downstream primer for amplifying the ETH10 gene shown in SEQ ID NO: 8;
a nucleotide sequence of an upstream primer for amplifying CSM0113 gene shown in SEQ ID NO: 9, and a nucleotide sequence of a downstream primer for amplifying the CSM0113 gene shown in SEQ ID NO: 10;
a nucleotide sequence of an upstream primer for amplifying INRA005 gene shown in SEQ ID NO: 11, and a nucleotide sequence of a downstream primer for amplifying the INRA005 gene shown in SEQ ID NO: 12;
a nucleotide sequence of an upstream primer for amplifying BT54 gene shown in SEQ ID NO: 13, and a nucleotide sequence of a downstream primer for amplifying the BT54 gene shown in SEQ ID NO: 14;
a nucleotide sequence of an upstream primer for amplifying BT61 gene shown in SEQ ID NO: 15, and a nucleotide sequence of a downstream primer for amplifying the BT61 gene shown in SEQ ID NO: 16;
a nucleotide sequence of an upstream primer for amplifying INRA023 gene shown in SEQ ID NO: 17, and a nucleotide sequence of a downstream primer for amplifying the INRA023 gene shown in SEQ ID NO: 18;
a nucleotide sequence of an upstream primer for amplifying BM2113 gene shown in SEQ ID NO: 19, and a nucleotide sequence of a downstream primer for amplifying the BM2113 gene shown in SEQ ID NO: 20;
a nucleotide sequence of an upstream primer for amplifying G18833 gene shown in SEQ ID NO: 21, and a nucleotide sequence of a downstream primer for amplifying the G18833 gene shown in SEQ ID NO: 22;
a nucleotide sequence of an upstream primer for amplifying UMN0929 gene shown in SEQ ID NO: 23, and a nucleotide sequence of a downstream primer for amplifying the UMN0929 gene shown in SEQ ID NO: 24; and
a nucleotide sequence of an upstream primer for amplifying INRA063 gene shown in SEQ ID NO: 25, and a nucleotide sequence of a downstream primer for amplifying the INRA063 gene shown in SEQ ID NO: 26.

2. A use of the composite primer set for identifying polymorphic markers of cattle according to claim 1, comprising:
performing species identification, individual identification, and kinship analysis on cattle by using the composite primer set for identifying polymorphic markers of cattle.

3. A kit for identifying polymorphic markers of cattle, comprising: the composite primer set for identifying polymorphic markers of cattle according to claim 1.

4. The kit according to claim 3, wherein the kit is used to perform species identification, individual identification, and kinship analysis on cattle.

5. A method for detecting 13 short tandem repeat (STR) loci of cattle using the primer set according to claim 1, comprising:
using the primer set in a polymerase chain reaction (PCR) system to perform multiplex PCR amplification on cattle genomic deoxyribonucleic acid (DNA) samples to obtain PCR amplification products, and performing genotyping detection on the PCR amplification products.

6. The method according to claim 5, wherein final reaction concentrations of each pair of the primers in the primer set are as follows:
the upstream primer and the downstream primer of the TGLA126 gene each is 1.5 micromoles per liter (μM);
the upstream primer and the downstream primer of the BT66 gene each is 0.6 μM;
the upstream primer and the downstream primer of the BT165 gene each is 0.6 μM;
the upstream primer and the downstream primer of the ETH10 gene each is 1 μM;
the upstream primer and the downstream primer of the CSM0113 gene each is 2.5 μM;
the upstream primer and the downstream primer of the INRA005 gene each is 2.5 μM;
the upstream primer and the downstream primer of the BT54 gene each is 1.5 μM;
the upstream primer and the downstream primer of the BT61 gene each is 2.25 μM;
the upstream primer and the downstream primer of the INRA023 gene each is 2.5 μM;
the upstream primer and the downstream primer of the BM2113 gene each is 3 μM;
the upstream primer and the downstream primer of the G18833 gene each is 2.5 μM;
the upstream primer and the downstream primer of the UMN0929 gene each is 4 μM; and
the upstream primer and the downstream primer of the INRA063 gene each is 5 μM.

7. The method according to claim 5, wherein the PCR system further comprises: 2×Multiplex PCR Master Mix, 5×Q-Solution, 10× the primers in the primer set, deionized water, and the cattle DNA samples.

8. The method according to claim 5, wherein reaction conditions of the multiplex PCR amplification comprise: pre-denaturation at 95 degrees Celsius (C) for 15 minutes; denaturation at 94° C. for 30 seconds, annealing at 57° C. for 90 seconds, extension at 72° C. for 90 seconds, repeat for 30 cycles; then final extension at 60° C. for 60 minutes, and heat preservation at 4° C.

9. The method according to claim 5, wherein at least one primer in each pair of the primers in the primer set is labeled with a fluorescent dye and the fluorescent dye is selected from a group consisting of 6-carboxyfluorescein (6FAM), hexachlorofluorescein (HEX), carboxytetramethylrhodamine TAMRA, and carboxy-X-rhodamine (ROX).

10. The method according to claim 5, wherein the method is applied for species identification, individual identification, and kinship analysis on cattle.

11. A method for detecting 13 STR loci of cattle using the kit according to claim 3, comprising:
using the primer set in a PCR system to perform a multiplex PCR amplification on cattle DNA samples to obtain PCR amplification products, and performing genotyping detection on the PCR amplification products.

12. The method according to claim 11, wherein final reaction concentrations of each pair of primers in the primer set are as follows:
the upstream primer and the downstream primer of the TGLA126 gene each is 1.5 μM;
the upstream primer and the downstream primer of the BT66 gene each is 0.6 μM;
the upstream primer and the downstream primer of the BT165 gene each is 0.6 μM;
the upstream primer and the downstream primer of the ETH10 gene each is 1 μM;
the upstream primer and the downstream primer of the CSM0113 gene each is 2.5 HM;
the upstream primer and the downstream primer of the INRA005 gene each is 2.5 μM;
the upstream primer and the downstream primer of the BT54 gene each is 1.5 μM;
the upstream primer and the downstream primer of the BT61 gene each is 2.25 M;
the upstream primer and the downstream primer of the INRA023 gene each is 2.5 μM;
the upstream primer and the downstream primer of the BM2113 gene each is 3 μM;
the upstream primer and the downstream primer of the G18833 gene each is 2.5 μM;
the upstream primer and the downstream primer of the UMN0929 gene each is 4 μM; and
the upstream primer and the downstream primer of the INRA063 gene each is 5 μM.

13. The method according to claim 11, wherein the PCR system further comprises:
2×Multiplex PCR Master Mix, 5×Q-Solution, 10×the primers in the primer set, deionized water, and the cattle DNA samples.

14. The method according to claim 11, wherein reaction conditions of the multiplex PCR amplification comprise: pre-denaturation at 95° C. for 15 minutes; denaturation at 94° C. for 30 seconds, annealing at 57° C. for 90 seconds, extension at 72° C. for 90 seconds, repeat for 30 cycles; then final extension at 60° C. for 60 minutes, and heat preservation at 4° C.

15. The method according to claim 11, wherein at least one primer in each pair of primers in the primer set is labeled with a fluorescent dye and the fluorescent dye is selected from a group consisting of 6FAM, HEX, TAMRA, and ROX.


SEQUENCE LISTING (26 sequences)

SEQ ID NO: 1 - DNA, 26 bp, synthetic construct
  source 1..26: mol_type = other DNA
ttggtcctctattctctgaatattcc

SEQ ID NO: 2 - DNA, 25 bp, synthetic construct
  source 1..25: mol_type = other DNA
ctaatttagaatgagagaggcttct

SEQ ID NO: 3 - DNA, 22 bp, synthetic construct
  source 1..22: mol_type = other DNA
gcagcatttctttggctgtaaa

SEQ ID NO: 4 - DNA, 18 bp, synthetic construct
  source 1..18: mol_type = other DNA
gtggttgcccgttcatta

SEQ ID NO: 5 - DNA, 18 bp, synthetic construct
  source 1..18: mol_type = other DNA
ggatcttcccgataaccc

SEQ ID NO: 6 - DNA, 19 bp, synthetic construct
  source 1..19: mol_type = other DNA
gtagcagcagtagcagttc

SEQ ID NO: 7 - DNA, 16 bp, synthetic construct
  source 1..16: mol_type = other DNA
acctgggtagtcggag

SEQ ID NO: 8 - DNA, 17 bp, synthetic construct
  source 1..17: mol_type = other DNA
gggaccatccattctgc

SEQ ID NO: 9 - DNA, 19 bp, synthetic construct
  source 1..19: mol_type = other DNA
agcaagacaggtgtttcaa

SEQ ID NO: 10 - DNA, 19 bp, synthetic construct
  source 1..19: mol_type = other DNA
cgactctggggatgatgta

SEQ ID NO: 11 - DNA, 17 bp, synthetic construct
  source 1..17: mol_type = other DNA
gggaatctgtggaggag

SEQ ID NO: 12 - DNA, 17 bp, synthetic construct
  source 1..17: mol_type = other DNA
caggcataccctacacc

SEQ ID NO: 13 - DNA, 20 bp, synthetic construct
  source 1..20: mol_type = other DNA
gtaagtgactgccagaaacc

SEQ ID NO: 14 - DNA, 19 bp, synthetic construct
  source 1..19: mol_type = other DNA
aacaggaaccagcatgaac

SEQ ID NO: 15 - DNA, 21 bp, synthetic construct
  source 1..21: mol_type = other DNA
taatgagcgagtgaaacaacg

SEQ ID NO: 16 - DNA, 21 bp, synthetic construct
  source 1..21: mol_type = other DNA
cagtgtaaacttgcctgtctt

SEQ ID NO: 17 - DNA, 22 bp, synthetic construct
  source 1..22: mol_type = other DNA
aactacagggtgttagatgaac

SEQ ID NO: 18 - DNA, 19 bp, synthetic construct
  source 1..19: mol_type = other DNA
caatgggtcagagtagagc

SEQ ID NO: 19 - DNA, 21 bp, synthetic construct
  source 1..21: mol_type = other DNA
tgtttatcatccaaggtgcca

SEQ ID NO: 20 - DNA, 19 bp, synthetic construct
  source 1..19: mol_type = other DNA
ctcccagatcaatccaaga

SEQ ID NO: 21 - DNA, 23 bp, synthetic construct
  source 1..23: mol_type = other DNA
gatcacttgagccaattccaata

SEQ ID NO: 22 - DNA, 20 bp, synthetic construct
  source 1..20: mol_type = other DNA
gcaacacctaccgtcttact

SEQ ID NO: 23 - DNA, 20 bp, synthetic construct
  source 1..20: mol_type = other DNA
accagctgatacacaagtgc

SEQ ID NO: 24 - DNA, 20 bp, synthetic construct
  source 1..20: mol_type = other DNA
ggtcagagaatgaaacagag

SEQ ID NO: 25 - DNA, 20 bp, synthetic construct
  source 1..20: mol_type = other DNA
tgtagctggcttacttacga

SEQ ID NO: 26 - DNA, 20 bp, synthetic construct
  source 1..20: mol_type = other DNA
aaggtgaccacaaagggatt